(19) United States
(12) Patent Application Publication
(10) Pub. No.: US 2022/0151494 A1
(43) Pub. Date: May 19, 2022

(54) MAGNETIC RESONANCE APPARATUS WITH A PATIENT VENTILATOR AND A METHOD FOR CONTROLLING A PATIENT VENTILATOR

(71) Applicant: Siemens Healthcare GmbH, Erlangen (DE)

(72) Inventor: Stephan Biber, Erlangen (DE)

(73) Assignee: Siemens Healthcare GmbH, Erlangen (DE)

(21) Appl. No.: 17/529,380

(22) Filed: Nov. 18, 2021

(30) Foreign Application Priority Data
Nov. 18, 2020 (DE) 102020214467.2

(51) Int. Cl. A61B 5/00; G01R 33/28; A61B 5/055; A61F 7/00

(52) U.S. Cl. CPC A61B 5/0036; G01R 33/288; A61F 2007/0064; A61F 7/00; A61B 5/055

(57) ABSTRACT

A magnetic resonance (MR) apparatus may include a patient ventilator, a cooler, and at least one electronic component. The patient ventilator may include at least one airflow generator configured to generate an airflow and at least one ventilation duct. The cooler may be configured to cool the at least one electronic component. The cooler may include at least one cooling element coupled to the patient ventilator.

### CROSS REFERENCE TO RELATED APPLICATIONS

This patent application claims priority to German Patent Application No. 10 2020 214 467.2, filed Nov. 18, 2020, which is incorporated herein by reference in its entirety.

### BACKGROUND

### Field

The present disclosure relates to a magnetic resonance apparatus, which comprises a patient ventilator, wherein the patient ventilator has at least one element generating an airflow and at least one ventilation duct, at least one electronic component and a cooler for cooling the at least one electronic component. Furthermore, the disclosure starts from a method for controlling a patient ventilator by means of a ventilation controller during a magnetic resonance examination.

### Related Art

Magnetic resonance apparatuses frequently have a very distributed cooling system. It can be the case that different components for cooling, such as an electronic receiving device of a radio frequency coil and/or an electronic emitting device of a radio frequency coil and/or further electronic components in each case have a separate cooling infrastructure. However, this leads to high costs and a high space requirement for the cooling system and its individual cooling elements.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a magnetic resonance apparatus with a patient ventilator according to an exemplary embodiment.

FIG. 2 shows the patient ventilator together with a cooler according to an exemplary embodiment.

FIG. 3 is a flowchart of a method for controlling the patient ventilator according to an exemplary embodiment.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to provide a compact and component-saving cooling system.

In an exemplary embodiment of the disclosure, a magnetic resonance apparatus includes:
a patient ventilator, wherein the patient ventilator has at least one element generating an airflow and at least one ventilation duct,
at least one electronic component and
a cooler for cooling the at least one electronic component,

wherein the cooler has at least one cooling element, which is coupled to the patient ventilator.

The magnetic resonance apparatus preferably comprises a medical and/or diagnostic magnetic resonance apparatus, which is configured and/or designed for acquiring medical and/or diagnostic image data, in particular medical and/or diagnostic magnetic resonance image data, of a patient. The magnetic resonance apparatus preferably comprises a scanner, in particular a magnetic unit, for the acquisition of the medical and/or diagnostic image data. Here the scanner, in particular the magnetic unit, comprises a basic magnet, a gradient coil and a radio frequency antenna. The radio frequency antenna is arranged permanently inside the scanner here. The local radio frequency coil is arranged around the region to be examined of the patient for acquisition of magnetic resonance signals.

For a magnetic resonance examination the patient, in particular the region to be examined of the patient, is positioned inside a patient receiving area of the magnetic resonance apparatus. The patient receiving area is at least partially surrounded by the scanner, in particular is cylindrically surrounded by the scanner. A Field of View (FOV) and/or an isocenter of the magnetic resonance apparatus is preferably arranged inside the patient receiving area. The FOV preferably comprises an acquisition region of the magnetic resonance apparatus, inside which the conditions for an acquisition of medical image data, in particular magnetic resonance image data, exist inside the patient receiving area, such as a homogeneous basic magnetic field. The isocenter of the magnetic resonance apparatus preferably comprises the region and/or point inside the magnetic resonance apparatus, which has the optimum and/or ideal conditions for the acquisition of medical image data, in particular magnetic resonance image data. In particular, the isocenter comprises the most homogeneous magnetic field region inside the magnetic resonance apparatus.

The magnetic resonance apparatus has a patient supporting apparatus for a positioning of the patient, in particular of the region to be examined of the patient, inside the patient receiving area. The patient supporting apparatus is designed for supporting the patient. The patient supporting apparatus preferably has a movable patient table, which is designed to be movable in particular inside the patient receiving area of the magnetic resonance apparatus. The patient is moved, together with the patient table, into the patient receiving area for a magnetic resonance examination.

The patient ventilator is configured and/or designed for ventilation of the patient receiving area during a magnetic resonance examination and/or during the time a patient spends inside the patient receiving area. The patient ventilator has for this purpose at least one element generating an airflow, such as a fan and/or ventilator. The element generating the airflow and/or a drive element for the generation of a drive moment for the element generating the airflow is preferably arranged on the scanner, preferably on a basic magnet and/or in the vicinity of the basic magnet of the scanner.

In addition, the patient ventilator has at least one ventilation duct. The ventilation duct is preferably configured and/or designed to transport the airflow generated by the element generating the airflow into the patient receiving area. In an exemplary embodiment, a casing surrounding the patient receiving area, for example the radio frequency coil, has a ventilation opening here for an inflow of the airflow into the patient receiving area. In addition, the patient ventilator can have a further ventilation duct, which is configured and/or designed to transport air to the element generating the airflow, such as an air intake duct.

The at least one electronic component preferably comprises an electronic component of the magnetic resonance apparatus, which is arranged in the vicinity of the scanner of the magnetic resonance apparatus. In particular, the at least one electronic component comprises an electronic component, which is arranged inside a housing of the scanner and/or is arranged inside a region surrounded by the housing of the scanner. The at least one electronic component can comprise an electronic emitting device for emitting a radio frequency pulse of the radio frequency antenna and/or an electronic receiving device for receiving a magnetic resonance signal of the radio frequency antenna and/or a local radio frequency coil. In addition, the at least one electronic component can also comprise an electronic control device and/or an electronic communications device and/or an electronic temperature-monitoring device and/or further electronic components that appear expedient to a person skilled in the art.

The cooler is configured for cooling the at least one electronic component. For this purpose, the cooler preferably has a cooling element or even a plurality of cooling elements. The at least one cooling element is designed to be coupled to the patient ventilator. In particular, the at least one cooling element of the cooler is coupled to the patient ventilator for cooling of the at least one electronic component. In an exemplary embodiment, the at least one cooling element of the cooler is designed as a cooling duct, which is coupled to the patient ventilator, in particular to the at least one ventilation duct of the patient ventilator and/or to the at least one element generating an airflow. In addition, the cooler can have even further cooling elements, such as a cooling element arranged on the at least one electronic component, such as, in particular, cooling fins, via which waste heat of the at least one electronic component can be given off to the surroundings and/or an ambient atmosphere.

In this way, the at least one electronic component can be particularly advantageously cooled. In particular, in this way, cooling of the at least one electronic component by means of an existing unit, in particular the patient ventilator, can be assisted. Particularly advantageously, an airflow for patient ventilation generated by the patient ventilator can also be used here for cooling the at least one electronic component. This contributes in particular to a reduction of costs and/or installation space. In an exemplary embodiment, a plurality of electronic components of the magnetic resonance apparatus, in particular electronic components which are surrounded by a housing of the scanner, can also be supplied by the patient ventilator for the purpose of cooling.

In an advantageous development of the inventive magnetic resonance apparatus it can be provided that the at least one ventilation duct of the patient ventilator has at least one airflow divider element, wherein the at least one cooling element of the cooler is coupled by means of the at least one airflow divider element to the patient ventilator. The at least one airflow divider element can be designed to be Y-shaped or also T-shaped. By means of the at least one airflow divider element, preferably a portion of the generated airflow of the patient ventilator is diverted into the at least one cooling element of the cooler and can therewith advantageously be fed to the at least one electronic component for cooling. In an exemplary embodiment, the at least one cooling element is designed here as a cooling duct, which is coupled by means of the airflow divider element to the at least ventilation duct. In this way, particularly simple utilization and/or provision of the patient ventilator, in particular an airflow produced and/or generated by the patient ventilator, can be enabled for the cooler, in particular for cooling the at least one electronic component.

In an advantageous development of the inventive magnetic resonance apparatus it can be provided that the at least one cooling element has a cooling duct and the at least one cooling duct comprises at least one further airflow divider element. The at least one further airflow divider element can also be designed to be Y-shaped or also T-shaped. By means of the at least one further airflow divider element, preferably a portion of the airflow introduced into the cooling duct is diverted into a further cooling duct of the cooler and can therewith advantageously be fed to at least one further electronic component for cooling. Advantageously, a plurality of electronic components can be easily and inexpensively cooled hereby.

In an advantageous development of the inventive magnetic resonance apparatus it can be provided that the at least one cooling element of the cooler is coupled at an air outlet side of the airflow generating element to the patient ventilator. The element generating the airflow, in particular a fan and/or ventilator, generates the airflow in a defined direction. The airflow is drawn in at a first side, in particular at an intake side, of the element generating the airflow and blown out and/or given off at a second side, in particular at an air outlet side, of the element generating the airflow. The element generating the airflow can be arranged inside the at least one ventilation duct. In addition, it may also be that the patient ventilator has at least two ventilation ducts. A first ventilation duct is designed to feed air to the element generating the airflow and is thus arranged at the intake side of the element generating the airflow. Ambient air can thus be drawn in by means of the first ventilation duct. A second ventilation duct, by contrast, is designed to divert air from the element generating the airflow and is thus arranged at the air outlet side of the element generating the airflow. The second ventilation duct at the air outlet side of the patient ventilator preferably ends in the patient receiving area for ventilation and/or cooling of the patient and/or of the patient receiving area. For this purpose, the patient receiving area and/or a housing surrounding the patient receiving area preferably has a ventilation opening at which the second ventilation duct ends in the patient receiving area. In this way, advantageous ventilation of the patient receiving area with simultaneous cooling of the at least one electronic component can be achieved. In addition, an inflow of heated air, in particular of waste heat of the at least one electronic component, or of dust, as can occur with an arrangement and/or a coupling of the cooling element of the cooler at the intake side of the element generating the airflow, can advantageously also be prevented in this way.

Basically, an arrangement and/or coupling of the at least one cooling element of the cooler to the patient ventilator at an intake side of the element generating the airflow is also possible, however. Furthermore, it may also be that the cooler has two or more cooling elements, in particular cooling elements designed as cooling ducts, with at least a first one of the plurality of cooling ducts being coupled at the intake side of the element generating the airflow to the patient ventilator for cooling at least one electronic component and at least one second one of the plurality of cooling ducts being coupled at the air outlet side of the element generating the airflow to the patient ventilator for cooling at least one electronic component.

In an advantageous development of the inventive magnetic resonance apparatus it can be provided that the at least one ventilation duct comprises a diameter of at least 5 mm and at most 100 mm. In an exemplary embodiment, the at least one ventilation duct has a diameter of at least 5 mm and at most 90 mm. In an exemplary embodiment, the at least one ventilation duct has a diameter of at least 5 mm and at most 80 mm. In an exemplary embodiment, the at least one ventilation duct has a diameter of at least 5 mm and at most 70 mm. In an exemplary embodiment, the at least one ventilation duct has a diameter of at least 5 mm and at most 60 mm. In an exemplary embodiment, the at least one ventilation duct has a diameter of at least 5 mm and at most 50 mm. In an exemplary embodiment, the at least one ventilation duct has a diameter of at least 5 mm and at most 40 mm. In this way, a particularly compact and space-saving patient ventilator can be provided.

In an advantageous development of the inventive magnetic resonance apparatus it can be provided that the at least one electronic component comprises an electronic emitting device and/or an electronic receiving device. In an exemplary embodiment, the at least one electronic component comprises an electronic emitting device for emitting a radio frequency signal and/or a radio frequency pulse of the radio frequency antenna. Furthermore, the at least one electronic component can comprise an electronic receiving device for receiving magnetic resonance signals, by means of a local radio frequency coil, which is arranged around the region to be examined of the patient. Advantageous cooling of the electronic emitting device and/or the electronic receiving device can be achieved hereby with low expenditure by utilizing the patient ventilator. In addition, the magnetic resonance apparatus can in this way also have a particularly compact and component-sparing construction since additional elements generating cooling can be omitted.

Alternatively or in addition, the at least one electronic component can comprise further electronic components, which are arranged in the vicinity of the patient ventilator and/or inside a region enclosed by a housing of the scanner. For example, a further electronic component can comprise an electronic control device and/or an electronic communications device and/or an electronic temperature-monitoring device, etc.

In an advantageous development of the inventive magnetic resonance apparatus it can be provided that the at least one ventilation duct and/or the at least one cooling element has at least one airflow regulator. The at least one airflow regulator can comprise a type of baffle, which is arranged inside the at least one ventilation duct and/or the at least one cooling element, in particular a cooling element designed as a cooling duct. In an exemplary embodiment, the at least one airflow regulator, in particular a position and/or an inclination of the at least one airflow regulator, is designed so that it can be set and/or controlled. In this way, an airflow for cooling and/or ventilation in the at least ventilation duct and/or in the at least one cooling element, in particular a cooling element designed as a cooling duct, can be set as a function of a position, in particular an inclination and/or a setting, of the at least one airflow regulator.

In an advantageous development of the inventive magnetic resonance apparatus it can be provided that the patient ventilator has a ventilation controller for control and/or regulation of the airflow as a function of at least one ventilation parameter. For example, the airflow can be controlled and/or regulated by control and/or setting an angle of inclination and/or tilt angle of the at least one airflow regulator by means of the ventilation controller. Furthermore, the airflow can also be controlled and/or regulated by control and/or setting of the element generating the airflow by means of the ventilation controller. The at least one ventilation parameter can be automatically acquired and supplied to the ventilation controller, such as a parameter and/or value of a sensor and/or an operating parameter. In addition, it may also be that the at least one ventilation parameter can be manually set and/or provided, such as an air supply of the patient receiving area, which the patient or a user, in particular a medical operator, can set themselves.

In an exemplary embodiment, the ventilation controller includes at least one computer and/or processing circuitry (e.g. a processor), wherein the ventilation controller is configured to control and/or regulate the airflow as a function of at least one ventilation parameter. In an exemplary embodiment, the ventilation controller is configured to execute computer-readable instructions in order to implement the control and/or regulation of the airflow as a function of at least one ventilation parameter. In particular, the ventilation controller comprises a memory storage unit (memory), with computer-readable information being stored on the storage, with the ventilation controller being configured to load the computer-readable information from the memory and to execute the computer-readable information in order to implement control and/or regulation of the airflow as a function of at least one ventilation parameter.

The components of the ventilation controller can be designed for the most part in the form of software components. Basically, some of these components can also be implemented in the form of software-supported hardware components, for example FPGAs or the like, in particular when particularly fast calculations are involved. Similarly, the required interfaces can be designed as software interfaces, for example when it is only a matter of acquisition of data from other software components. However, they can also be designed as interfaces constructed in terms of hardware and which are actuated by suitable software. Of course, it is also conceivable that a plurality of said components are implemented in a combined manner in the form of an individual software component or software-supported hardware component.

This embodiment of the disclosure has the advantage that a simple and efficient regulation and/or control of the airflow and therewith also ventilation of the patient during a magnetic resonance examination and at the same time efficient cooling of the at least one electronic component can take place.

In an advantageous development of the inventive magnetic resonance apparatus it can be provided that the ventilation controller is configured and/or designed to control and/or regulate a speed of the element generating the airflow, in particular of the fan and/or the ventilator, as a function of at least one ventilation parameter. Simple and efficient regulation and/or control of the airflow and therewith also patient ventilation and/or cooling of the at least one electronic component can take place hereby.

In an advantageous development of the inventive magnetic resonance apparatus it can be provided that the at least one ventilation parameter comprises patient information and/or temperature information of the at least one electronic component and/or a cooling requirement of one of the at least one electronic components and/or an operating mode of the magnetic resonance apparatus. The patient information can comprise, for example, the duration of an examination of the magnetic resonance examination and/or position information of the patient inside the patient receiving area and/or a condition of the patient and/or a selected and/or set air supply during the magnetic resonance examination. The selected and/or set air supply during the magnetic resonance examination and/or further patient information can be set and/or selected by a patient and/or by a user, in particular the medical operator. For example, with a relatively long duration of an examination, a stronger ventilation flow can be set than with a shorter duration of an examination. In addition, it may also be that with a nervous patient, an airflow contributes to calming, etc.

The temperature information of the at least one electronic component can comprise, for example, a current temperature value, which the at least one electronic component has. The temperature information is preferably acquired by means of a temperature sensor, which is incorporated by the at least one electronic component. It may be that an all the greater and/or stronger cooling and/or cooling capacity for cooling the at least one electronic component is required, the higher the current temperature value is. A speed of the element generating the airflow and/or a setting of an airflow regulator can also be set thereby as a function of the temperature information of the at least one electronic component.

The cooling requirement of the at least one electronic component can comprise, for example, a maximum cooling requirement of the at least one electronic component. In addition, the cooling requirement of the at least one electronic component can also be determined on the basis of settings and/or parameters of the current magnetic resonance examination. First of all an anticipated heating of the at least one electronic component can be determined here and a cooling requirement can then be determined from this. The cooling requirement of the at least one electronic component can be automatically determined at least in part by means of the ventilation controller.

In addition, on the basis of an operating mode of the magnetic resonance apparatus, in particular of the scanner of the magnetic resonance apparatus, heating of the at least one electronic component can be determined and from this a cooling of the at least one electronic component and/or a ventilation of the patient receiving area can be set and/or controlled. An operating mode of this kind of the magnetic resonance apparatus can be, for example, “Scan Mode” or a “Ready to Scan Mode” or an “Eco Mode” or an “Off Mode”, etc. In particular, a speed of the element generating the airflow, in particular of the fan and/or of the ventilator, can be increased as soon as the magnetic resonance apparatus is in the “Scan Mode”. By contrast, the speed of the element generating the airflow, in particular of the fan and/or ventilator, can be reduced as soon as the magnetic resonance apparatus is in the “Eco Mode” or “Ready to Scan Mode”.

In this way, simple control and/or regulation of the speed of the element generating the airflow, in particular of the fan and/or ventilator, and/or simple control and/or regulation of a setting of the airflow regulator as a function of a current requirement, in particular of the patient and/or the electronic component for cooling and/or the magnetic resonance apparatus, can advantageously be achieved.

In an advantageous development of the inventive magnetic resonance apparatus it can be provided that the patient ventilator has at least one air heating element. The air heating element can advantageously comprise a heating element for heating the drawn-in air before the air flows into the patient receiving area. In an exemplary embodiment, the air heating element is arranged on and/or in the at least one ventilation duct in such a way that only the air flowing into the patient receiving area is heated and not a portion of the airflow provided for cooling. The air heating element can also be designed to be controllable, in particular as an air heating element which can be controlled by means of the ventilation controller.

Alternatively or in addition, it may also be that the air heating element is designed as at least one electronic component for cooling and the waste heat of the at least one electronic component for cooling is used in order to heat the airflow flowing into the patient receiving area. For this purpose, a cooling duct of the cooler for cooling the at least one electronic component preferably is coupled at the intake side of the element of the patient ventilator generating the airflow.

Such an embodiment of the disclosure has the advantage that the air flowing into the patient receiving area can be heated for ventilation of the patient receiving area and it is therewith advantageously possible to reduce and/or prevent the patient from becoming cold, in particular in the case of relatively long examination times and/or residence times of the patient inside the patient receiving area.

Furthermore, the disclosure starts from a method for controlling a patient ventilator by means of a ventilation controller during a magnetic resonance examination, wherein the patient ventilator is encompassed by a magnetic resonance apparatus, wherein the ventilation controller controls the patient ventilator as a function of a patient parameter and at least one further ventilation parameter, wherein the further ventilation parameter comprises a cooling parameter of a cooler of the magnetic resonance apparatus for cooling at least one electronic component.

In this way, ventilation of a patient receiving area of the magnetic resonance apparatus can be particularly advantageously achieved with simultaneous cooling of the at least one electronic component. In particular, cooling of the at least one electronic component by means of an existing unit can be supported in this way. This contributes, in particular, to a reduction in costs and/or components and/or installation space. In an exemplary embodiment, a plurality of electronic components of the magnetic resonance apparatus, in particular electronic components which are surrounded by a housing of the scanner, are also supplied by the patient ventilator here for the purpose of cooling.

The advantages of the inventive method for controlling a patient ventilator by means of a ventilation controller during a magnetic resonance examination substantially correspond to the advantages of the inventive magnetic resonance apparatus, which have been stated above in detail. Features, advantages or alternative embodiments mentioned in this connection can likewise also be transferred to the other claimed subject matters, and vice versa.

In an advantageous development of the inventive method it can be provided that the at least one further ventilation parameter comprises an operating mode of the magnetic resonance apparatus and/or temperature information of the at least one electronic component for cooling by means of the patient ventilator and/or a maximum cooling requirement of the at least one electronic component for cooling by means of the patient ventilator. This embodiment of the disclosure has the advantage that a simple and efficient regulation and/or control of the airflow for ventilation of the patient receiving area and/or for cooling the at least one electronic component can take place.

In an advantageous development of the inventive method it can be provided that a position and/or a setting of at least one airflow regulator can be set by means of the ventilation controller. In an exemplary embodiment, the at least one airflow regulator is arranged inside a ventilation duct of the patient ventilator. In addition, the at least one airflow regulator can also be arranged inside a cooling duct, which is configured and/or designed for feeding cooling air to the electronic component for cooling. Simple control and/or setting of the airflow for cooling of the patient ventilator can advantageously be achieved by the control and/or regulation of a setting and/or a position of the airflow regulator as a function of a current requirement, in particular of the patient and/or the electronic component for cooling and/or the magnetic resonance apparatus.

In an advantageous development of the inventive method it can be provided that a speed of at least one element generating the airflow can be set by means of the ventilation controller. By means of the ventilation controller, an intensity of the airflow for cooling the at least one electronic component and/or for ventilation of the patient receiving area can advantageously be controlled and/or set by the ventilation controller. In particular, simple control and/or setting of the speed of the element generating the airflow, in particular of the fan and/or ventilator, can advantageously be achieved as a function of a current requirement, in particular of the patient and/or the electronic component for cooling and/or the magnetic resonance apparatus.

In an advantageous development of the inventive method it can be provided that at least one parameter for setting and/or control of the patient ventilator can be set by a user, in particular a medical operator. For example, an air supply for the patient can be fixed by the user. In this way, at least one parameter, for example the patient parameter, can be individually adjusted.

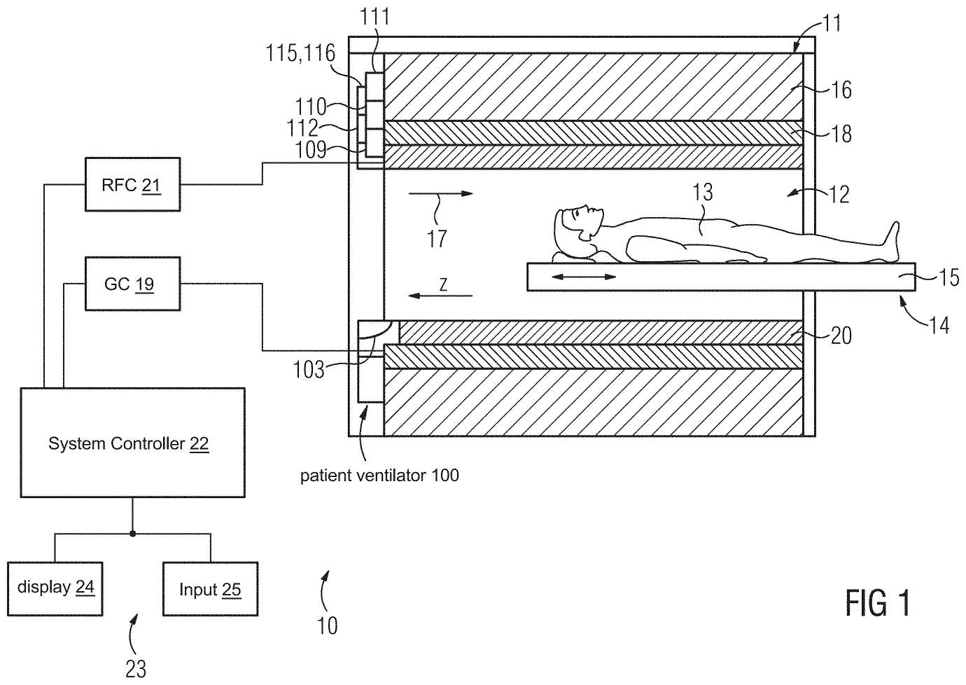

FIG. 1 schematically represents a magnetic resonance apparatus 10. The magnetic resonance apparatus 10 comprises a scanner 11 formed by a magnetic unit. In addition, the magnetic resonance apparatus 10 has a patient receiving area 12 for receiving a patient 13. In the present exemplary embodiment, the patient receiving area 12 is cylindrical and cylindrically surrounded in a circumferential direction by the scanner 11, in particular by the magnetic unit. Basically, a different design of the patient receiving area 12 is always conceivable, however. The patient 13 can be pushed and/or moved by means of a patient supporting apparatus 14 of the magnetic resonance apparatus 10 into the patient receiving area 12. The patient supporting apparatus 14 has for this purpose a patient table 15 configured to be movable inside the patient receiving area 12. In particular, the patient table 15 is movably mounted in the direction of a longitudinal extension of the patient receiving area 12 and/or in the z-direction here.

The scanner 11, in particular the magnetic unit, comprises a superconducting basic magnet 16 for generating a strong and, in particular, constant basic magnetic field 17. Furthermore, the scanner 11, in particular the magnetic unit, has a gradient coil 18 for generation of magnetic field gradients, which are used for spatial encoding during imaging. The gradient coil 18 is controlled by means of a gradient controller 19 of the magnetic resonance apparatus 10. The scanner 11, in particular the magnetic unit, also comprises a radio frequency antenna 20 for excitation of a polarization, which establishes itself in the basic magnetic field 17 generated by the basic magnet 16. The radio frequency antenna 20 is controlled by a radio frequency antenna controller 21 of the magnetic resonance apparatus 10 and irradiates radio frequency magnetic resonance sequences into the patient receiving area 12 of the magnetic resonance apparatus 10.

The magnetic resonance apparatus 10 has a system controller 22 configured to control the basic magnet 16, the gradient controller 19, and/or the radio frequency antenna controller 21. The system controller 22 centrally controls the magnetic resonance apparatus, such as carrying out a predetermined imaging gradient echo sequence. In addition, the system controller 22 comprises an evaluator (not shown in more detail) configured to evaluate medical image data, which is acquired during the magnetic resonance examination. In an exemplary embodiment, the controller 22 includes processing circuitry that is configured to perform one or more functions and/or operations of the controller 22.

Furthermore, the magnetic resonance apparatus 10 comprises a user interface 23, which is connected to the system controller 22. Control information such as imaging parameters, as well as reconstructed magnetic resonance images, can be displayed on a display 24, for example on at least one monitor, of the user interface 23, for a medical operator. Furthermore, the user interface 23 has an input 25 by means of which information and/or parameters can be input during a measuring process by the medical operator.

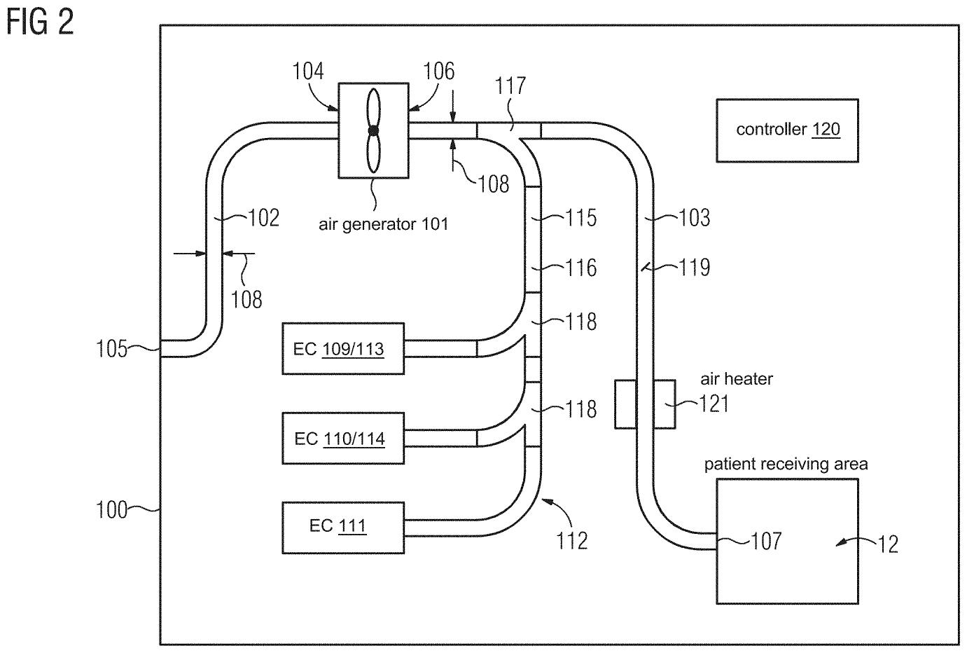

The magnetic resonance apparatus 10 also comprises a patient ventilator 100 (FIGS. 1 and 2) for a ventilation of the patient receiving area 12 during a magnetic resonance examination of a patient 13. The patient ventilator 100 is shown in more detail in FIG. 2 and has at least one element (airflow generator) 101 generating the airflow and at least one ventilation duct 102, 103. In the present exemplary embodiment, the patient ventilator 100 has a single airflow generator 101 configured to generate an airflow. In an alternative embodiment of the disclosure, the patient ventilator 100 can also have two or more elements (airflow generators) 101 configured to generate an airflow. In an exemplary embodiment, the airflow generator 101 is formed by a fan and/or a ventilator. In an exemplary embodiment, the airflow generator 101 generating the airflow and/or a drive element of the patient ventilator 100 configured to generate a drive moment for the airflow generator 101 generating the airflow is arranged on the scanner 11, such as on the basic magnet 16 and/or in the vicinity of the basic magnet 16 of the scanner 11. In an exemplary embodiment, the patient ventilator 100 (and/or one or more components of the patient ventilator 100) includes processing circuitry that is configured to perform one or more functions and/or operations of the patient ventilator 100 (or respective functions and/or operations of the component(s)).

In an exemplary embodiment, the patient ventilator 100 has two ventilation ducts 102, 103, with a first ventilation duct 102 of the two ventilation ducts 102, 103 being designed for drawing in air and a second ventilation duct 103 of the two ventilation ducts 102, 103 being designed for blowing out air. The first ventilation duct 102 is thus arranged at an intake side 104 of the airflow generator 101 generating the airflow, in particular of the fan and/or of the ventilator, so that an airflow in the first ventilation duct 102 flows toward the airflow generator 101 generating the airflow, in particular toward the fan and/or ventilator. The first ventilation duct 102 has an intake opening 105 drawing in air, which is arranged on a housing 26 of the magnetic resonance apparatus 10 surrounding the scanner 11. The second ventilation duct 103 is arranged at an air outlet side 106 of the airflow generator 101 generating the airflow, in particular of the fan and/or ventilator, so that an airflow in the second ventilation duct 103 flows away from the airflow generator 101 generating the airflow, in particular the fan and/or ventilator. The second ventilation duct 103 has an exhaust opening 107, which ends in the patient receiving area 12.

The first ventilation duct 102 and/or the second ventilation duct 103 has a diameter 108 of at least 5 mm and at most 100 mm. In an exemplary embodiment, the first ventilation duct 102 and/or the second ventilation duct 103 has a diameter 108 of at least 5 mm and at most 90 mm. In an exemplary embodiment, the first ventilation duct 102 and/or the second ventilation duct 103 has a diameter 108 of at least 5 mm and at most 80 mm. In an exemplary embodiment, the first ventilation duct 102 and/or the second ventilation duct 103 has a diameter 108 of at least 5 mm and at most 70 mm. In an exemplary embodiment, the first ventilation duct 102 and/or the second ventilation duct 103 has a diameter 108 of at least 5 mm and at most 60 mm. In an exemplary embodiment, the first ventilation duct 102 and/or the second ventilation duct 103 has a diameter 108 of at least 5 mm and at most 50 mm. In an exemplary embodiment, the first ventilation duct 102 and/or the second ventilation duct 103 has a diameter 108 of at least 5 mm and at most 40 mm.

In an exemplary embodiment, the magnetic resonance apparatus 10 has at least one electronic component 109, 110, 111 and a cooler 112 for cooling the at least one electronic component 109, 110, 111. The at least one electronic component 109, 110, 111 is arranged on the scanner 11 and/or is surrounded by the housing 26 of the scanner 11, so that the at least one electronic component 109, 110, 111 for cooling is arranged in the vicinity of the patient ventilator 100. In an exemplary embodiment, the at least one electronic component 109, 110, 111 comprises an electronic emitting device (transmitter) 113 configured to emit a radio frequency (RF) pulse, an electronic receiver 114 configured to receive a magnetic resonance signal, and a further electronic component 111. In an exemplary embodiment, the further electronic component 111 is an electronic controller, an electronic communications device, an electronic temperature-monitor, and/or one or more other electronic components as would be understood by one of ordinary skill in the art.

In an alternative embodiment of the disclosure, the magnetic resonance apparatus 10 can also comprise more than one further electronic component 111. Furthermore, in an alternative embodiment of the disclosure, the at least one electronic component 109, 110, 111 can also comprise just one electronic emitting device 113 or just one electronic receiving device 114 or just one further electronic component 111 or any combination whatsoever of the electronic components 109, 110, 111.

The cooler 112 has at least one cooling element 115, which is formed by a cooling duct 116 for feeding a cooling fluid, such as air, for cooling the at least one electronic component 109, 110, 111. The cooling element 115, in particular the cooling duct 116, is coupled here to the patient ventilator 100. In the present exemplary embodiment, the cooling element 115, in particular the cooling duct 116, is coupled at the air outlet side 106 of the airflow generator 101 generating the airflow, in particular of the fan and/or of the ventilator, to the patient ventilator 100. In an exemplary embodiment, the cooling element 115, in particular the cooling duct 116, is coupled here to the second ventilation duct 103 of the patient ventilator 100. In an alternative embodiment of the cooler 112 and/or the patient ventilator 100, the cooling element 115, in particular the cooling duct 116, of the cooler 112, can also be coupled at an intake side 104 of the airflow generator 101 generating the airflow, in particular of the fan and/or the ventilator, to the first ventilation duct 102 of the patient ventilator 100.

For coupling of the cooling element 115, in particular of the cooling duct 116, of the cooler 112 to the ventilation duct 103, in particular the second ventilation duct 103 of the patient ventilator 100, the second ventilation duct 102 has an airflow divider element 117, with the cooling duct 116 being coupled to the second ventilation duct 103 by means of the airflow divider element 117, see FIGS. 1 and 2 in this regard. The airflow divider element 117 is Y-shaped and/or T-shaped and has an air inlet and two air outlets here. In addition, the cooling element 115, in particular the cooling duct 116, of the cooler 112 also has at least one further airflow divider element 118. In the present exemplary embodiment, the cooler 112 has two further airflow divider elements 118. The individual further airflow divider elements 118 are likewise Y-shaped and/or T-shaped respectively and each have one air inlet and two air outlets, as can be seen in FIG. 2. In this way, an airflow for cooling can be diverted for each electronic component 109, 110, 111.

Furthermore, the ventilation duct 103, in particular the second ventilation duct 103, of the patient ventilator 100 has an airflow regulator 119. In the present exemplary embodiment, the airflow regulator 119 comprises a type of baffle, which is arranged inside the second ventilation duct 103. The at least one airflow regulator 119 is designed to be settable and/or controllable, so that an airflow volume for ventilation in the second ventilation duct 103 can be set as a function of a position, in particular an inclination and/or a setting, of the at least one airflow regulator 119. Alternatively or in addition, the cooling element 115, in particular the cooling duct 116, of the cooler 112 can also have an airflow regulator 119.

By changing a position, in particular an inclination and/or a setting, of the airflow regulator 119, an effective cross-section 108 of the ventilation duct 103, in particular of the second ventilation duct 103, is changed and therewith also an airflow volume, which flows through the ventilation duct 103, in particular the second ventilation duct 103. For example, an airflow, in particular an airflow volume flowing through the second ventilation duct 103, can be reduced by reducing the cross-section 108 of the ventilation duct 103, in particular of the second ventilation duct 103, by the airflow regulator 119 reducing an effective cross-sectional area 108 of the ventilation duct 103, in particular the second ventilation duct 103. If, for example, the effective cross-sectional area corresponds identically or substantially to the cross-sectional area of the ventilation duct 103, in particular of the second ventilation duct 103, a surface normal of a regulating surface of the airflow regulator 119 is oriented substantially perpendicularly to the flow direction of the airflow in the ventilation duct 103, in particular the second ventilation duct 103. If, by contrast, the effective cross-sectional area corresponds identically or substantially to a minimum cross-sectional area of the ventilation duct 103, in particular of the second ventilation duct 103, a surface normal of a regulating surface of the airflow regulator 119 is oriented substantially parallel to the flow direction of the airflow in the ventilation duct 103, in particular the second ventilation duct 103.

The patient ventilator 100 also has a ventilation controller 120 for control and/or regulation of the airflow as a function of at least one ventilation parameter. In an exemplary embodiment, the ventilation controller 120 includes a computer and/or processing circuitry (e.g. a processor). In addition, the ventilation controller 120 comprises appropriate control software and/or appropriate control programs, which carry out control and/or regulation of the airflow of the patient ventilator 100 when executed by the computer and/or the processing circuitry. In an exemplary embodiment, the ventilation controller 120 includes processing circuitry that is configured to perform one or more functions and/or operations of the controller 120.

The at least one ventilation parameter can comprise patient information and/or temperature information of the at least one electronic component 109, 110, 111 for cooling and/or a cooling requirement of the at least one electronic component 109, 110, 111 for cooling and/or an operating mode of the magnetic resonance apparatus 10. The patient information can comprise, for example, a duration of an examination and/or position information of the patient 13 inside the patient receiving area 12 and/or a condition of the patient 13 and/or an air supply selected and/or set by the patient 13 during the magnetic resonance examination. The patient information can be retrieved at least partially automatically from the ventilation controller 120. In addition, it may also be, however, that the patient information is also manually input by a user, in particular a medical operator, by means of the user interface 23.

The temperature information of the at least one electronic component 109, 110, 111 can comprise, for example, a current temperature value, which has the at least one electronic component 109, 110, 111. The temperature information is preferably acquired by means of a temperature sensor (not shown in more detail) of the at least one electronic component 109, 110, 111 and supplied to the ventilation controller. For example, an all the higher and/or greater cooling and/or cooling capacity can be necessary for cooling the at least one electronic component 109, 110, 111 the higher the current temperature value of the at least one electronic component 109, 110, 111 is. The cooling requirement of the at least one electronic component 109, 110, 111 can comprise, for example, a maximum cooling requirement of the at least one electronic component 109, 110, 111. In addition, the cooling requirement of the at least one electronic component 109, 110, 111 can also be determined on the basis of settings and/or parameters of the current magnetic resonance examination. In this connection, firstly an anticipated heating of the at least one electronic component 109, 110, 111 can be ascertained and from this a cooling requirement of the at least one electronic component 109, 110, 111 can then be determined. The cooling requirement of the at least one electronic component 109, 110, 111 can be determined automatically by means of the ventilation controller 120.

The operating mode of the magnetic resonance apparatus 10 can comprise, for example, “Scan Mode” or a “Ready to Scan Mode” or an “Eco Mode” or an “Off Mode”. In particular, the airflow can be strengthened by means of the ventilation controller 120 as soon as the magnetic resonance apparatus 10 is in the “Scan Mode”. By contrast, the airflow can be reduced by means of the ventilation controller 120 as soon as the magnetic resonance apparatus 10 is in the “Eco Mode” or “Ready to Scan Mode”. The ventilation controller is connected in respect of a data exchange to the system controller 22 of the magnetic resonance apparatus 10 for the provision of the information of the operating mode of the magnetic resonance apparatus 10.

For regulation and/or control of the airflow, in particular of the airflow volume, as a function of at least one ventilation parameter, the ventilation controller 120 can be designed to control and/or regulate a speed of the airflow generator 101 generating the airflow, in particular of the fan and/or ventilator, as a function of the at least one ventilation parameter. With a high cooling requirement and/or high ventilation requirement, which results from the ventilation parameters, a speed of the airflow generator 101 generating the airflow, in particular of the fan and/or ventilator, can be set by the ventilation controller 120 in such a way that a large to maximum airflow volume is generated and flows through the second ventilation duct 103 and/or the cooling duct 16. If, by contrast, a low cooling requirement and/or low ventilation requirement exists, which results from the ventilation parameters, the ventilation controller 120 sets a speed of the airflow generator 101 generating the airflow, in particular of the fan and/or ventilator, in such a way that a small to minimal airflow volume is generated and is transported through the second ventilation duct 103 and/or the cooling duct 116.

Alternatively or in addition, for regulation and/or control of the airflow as a function of at least one ventilation parameter, the ventilation controller 120 can be designed to adjust and/or control a setting and/or position, in particular an inclination, of the at least one airflow regulator 119 here. With a high cooling requirement and/or high ventilation requirement, which results from the ventilation parameters, the airflow regulator 119 can be set by the ventilation controller 120 in such a way that large to maximum airflow flows through the second ventilation duct 103. If, by contrast, a low cooling requirement and/or low ventilation requirement exists, which results from the ventilation parameters, the ventilation controller 120 sets the airflow regulator 119 in such a way that small to minimal airflow flows through the second ventilation duct 103.

Furthermore, the patient ventilator 100 has an air heating element (heater) 121. In the present exemplary embodiment, the air heating element 121 comprises a heating element for heating the drawn-in air before the air flows into the patient receiving area 12. In an exemplary embodiment, the air heating element 121 is arranged on and/or in the second ventilation duct 103 in such a way that only the air flowing into the patient receiving area 12 is heated and not a portion of the airflow provided for cooling the at least one electronic component 109, 110, 111. In the present exemplary embodiment, the air heating element 121 is arranged on the second ventilation duct 103, in particular an outer side of the second ventilation duct 103. During operation of the air heating element 121 the second ventilation duct 103 is thus heated and therewith also the air flowing into the second ventilation duct 103. The air heating element 121 is also designed to be controllable. Heating of the airflow as a function of a set temperature parameter is controlled here by the ventilation controller 120. The temperature parameter is preferably manually communicated by the medical operator via the user interface 23 to the ventilation controller 120.

The represented magnetic resonance apparatus 10 can of course comprise further components which magnetic resonance apparatuses 10 conventionally have. A general mode of operation of a magnetic resonance apparatus 10 is known to a person skilled in the art, moreover, so that a detailed description of the further components will be omitted.

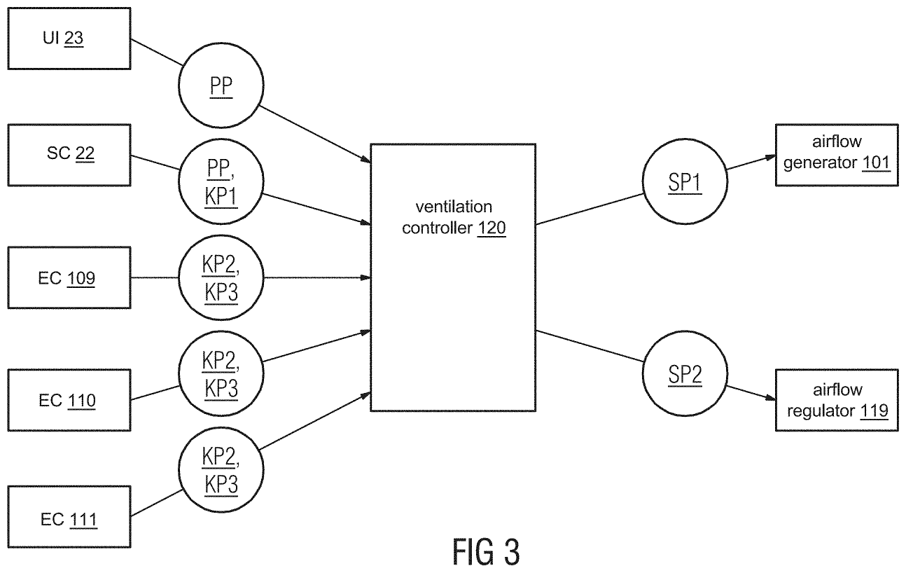

FIG. 3 represents an inventive method for controlling the patient ventilator 100 of the magnetic resonance apparatus 10 by means of the ventilation controller 120 during a magnetic resonance examination. The ventilation controller 120 controls the patient ventilator 100 as a function of at least one patient parameter PP and at least one further ventilation parameter, with the further ventilation parameter comprising a cooling parameter KP1, KP2, KP3 of the cooler of the magnetic resonance apparatus 10 for cooling the at least one electronic component 109, 110, 111. The patient parameter PP can be manually provided by a user interface 23 of the magnetic resonance apparatus 10. In addition, the patient parameter PP can also be provided in an at least partially automated manner by the system controller 22 of the magnetic resonance apparatus 10 of the ventilation controller 120.

At least one first further ventilation parameter, in particular a first cooling parameter KP1, can comprise an operating mode of the magnetic resonance apparatus 10. The ventilation controller 120 is connected to the magnetic resonance apparatus 10, in particular to the system controller 22, in respect of a data exchange, in particular an exchange of the patient parameter PP and/or of the first cooling parameter KP1.

A second further ventilation parameter, in particular a second cooling parameter KP2, can comprise temperature information of the at least one electronic component 109, 110, 111 for cooling by means of the patient ventilator 100. A third further ventilation parameter, in particular a third cooling parameter KP3, can comprise a maximum cooling requirement of the at least one electronic component 109, 110, 111 for cooling by means of the patient ventilator 100. The ventilation controller 120 retrieves the second and third cooling parameters KP2, KP3 automatically from the electronic components 109, 110, 111 for cooling. For this purpose, the ventilation controller is connected in respect of a data exchange to the electronic components 109, 110, 111 for cooling, in particular to the temperature sensors of the electronic components 109, 110, 111 for cooling.

Control parameters SP1, SP2 are generated inside the ventilation controller 120 here as a function of the patient parameter PP and the further ventilation parameters, in particular cooling parameters KP1, KP2, KP3. The ventilation controller 120 can control the airflow generator 101 generating the airflow, in particular the fan and/or ventilator, on the basis of the first control parameter SP1. In particular a speed of the airflow generator that generates the airflow is set and/or controlled here by the ventilation controller 120. Furthermore, a position and/or setting, in particular an inclination, of the airflow regulator 119 can be set by means of the ventilation controller 120 by means of the second control parameter SP2.

Although the disclosure has been illustrated and described in more detail by the preferred exemplary embodiment it is not restricted by the disclosed examples and a person skilled in the art can derive other variations herefrom without departing from the scope of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A magnetic resonance apparatus comprising:
a patient ventilator including at least one airflow generator and at least one ventilation duct;
at least one electronic component; and
a cooler configured to cool the at least one electronic component, wherein the cooler has at least one cooling element coupled to the patient ventilator.

2. The magnetic resonance apparatus as claimed in claim 1, wherein the at least one ventilation duct of the patient ventilator has at least one airflow divider, wherein the at least one cooling element of the cooler is coupled to the patient ventilator by the at least one airflow divider.

3. The magnetic resonance apparatus as claimed in claim 2, wherein the at least one cooling element includes a cooling duct having at least one further airflow divider.

4. The magnetic resonance apparatus as claimed in claim 1, wherein the at least one cooling element of the cooler is coupled at an air outlet side of the airflow generator to the patient ventilator.

5. The magnetic resonance apparatus as claimed in claim 1, wherein the at least one ventilation duct comprises a diameter of at least 5 mm and at most 100 mm.

6. The magnetic resonance apparatus as claimed in claim 1, wherein the at least one electronic component comprises an electronic transmitter and/or an electronic receiver.

7. The magnetic resonance apparatus as claimed in claim 1, wherein the at least one ventilation duct and/or the at least one cooler has at least one airflow regulator.

8. The magnetic resonance apparatus as claimed in claim 1, wherein the patient ventilator has a ventilation controller configured to control and/or regulate the airflow as a function of at least one ventilation parameter.

9. The magnetic resonance apparatus as claimed in claim 8, wherein the ventilation controller is configured to control and/or regulate a speed of the airflow generator as a function of the at least one ventilation parameter.

10. The magnetic resonance apparatus as claimed in claim 8, wherein the at least one ventilation parameter comprises:
patient information and/or temperature information of the at least one electronic component;
a cooling requirement of the at least one electronic component; and/or
an operating mode of the magnetic resonance apparatus.

11. The magnetic resonance apparatus as claimed in claim 1, wherein the patient ventilator has at least one air heater.

12. A method for controlling a patient ventilator by a ventilation controller during a magnetic resonance examination, the patient ventilator being encompassed by a magnetic resonance apparatus, the method comprising:
determining a patient parameter and a ventilation parameter; and
controlling, by the ventilation controller, the patient ventilator based on the patient parameter and the ventilation parameter, wherein the ventilation parameter comprises a cooling parameter of a cooler of the magnetic resonance apparatus for cooling at least one electronic component.

13. The method as claimed in claim 12, wherein the ventilation parameter comprises:
an operating mode of the magnetic resonance apparatus;
temperature information of the at least one electronic component for cooling by the patient ventilator; and/or
a maximum cooling requirement of the at least one electronic component for cooling by the patient ventilator.

14. The method as claimed in claim 12, further comprising setting a position and/or a setting of at least one airflow regulator by the ventilation controller.

15. The method as claimed in claim 12, further comprising setting a speed of an airflow generator by the ventilation controller.

16. The method as claimed in claim 12, further comprising manually setting, by a user, at least one parameter for setting and/or control of the patient ventilator.

17. A computer program which includes a program and is directly loadable into a memory of the magnetic resonance apparatus, when executed by a processor of the magnetic resonance apparatus, causes the processor to perform the method as claimed in claim 12.

18. A non-transitory computer-readable storage medium with an executable program stored thereon, that when executed, instructs a processor to perform the method of claim 12.
